(19) United States
(12) Patent Application Publication
(10) Pub. No.: US 2015/0351941 A1
(43) Pub. Date: Dec. 10, 2015

(54) PAIN FREE SOCKET SYSTEM AND ASSOCIATED METHOD

(71) Applicant: Katherine E. BOMKAMP, Colfax, NC (US)

(72) Inventor: Katherine E. BOMKAMP, Colfax, NC (US)

(21) Appl. No.: 14/377,642

(22) PCT Filed: Feb. 15, 2013
(86) PCT No.: PCT/US2013/026278

§ 371 Date: Aug. 8, 2014

Related U.S. Application Data
(60) Provisional application No. 61/599,464, filed on Feb. 16, 2012.

(51) Int. Cl. A61F 2/80; A61F 2/50

(52) U.S. Cl. CPC A61F 2/80; A61F 2/5044

(57) ABSTRACT

Systems and methods for providing adjustable heat to a prosthetic device are disclosed. A system includes: a temperature setting device configured to set a target temperature for the prosthetic device; an adjustable heating unit in a socket of the prosthetic device; a temperature sensor that detects an actual temperature in the socket of the prosthetic device; a controller that receives data from the temperature setting device and the temperature sensor, and that controls an amount of heat generated by the heating unit based on the data; and a display that displays the target temperature and the actual temperature.

### CROSS REFERENCE TO RELATED APPLICATIONS

This application claims priority to U.S. Provisional Patent Application No. 61/599,464, filed Feb. 16, 2012, the contents of which are incorporated by reference herein in their entirety.

### BACKGROUND

The present invention generally relates to a pain reducing and eliminating prosthesis socket device. More specifically, the invention is a pain reducing and eliminating prosthesis socket device that reduces and eliminates phantom pain.

Phantom Pain is a pain experienced by about 80% of the world's 10 million amputees. It is defined as the feeling of pain in a patient's non-existent limb, caused by the severed nerve endings at the site of amputation continuing to send pain signals and commands back to the brain.

Phantom pain is experienced differently by every person. The pain has been described as burning, itching, numbness, stabbing, cramping, etc. For some it is a merely annoying pain that lasts a couple of days or weeks after amputation, and for some the pain is debilitating that lasts for years. While the duration the pain is experienced depends on the person, research has shown that the longer a person experiences phantom pain, the more difficult it becomes to treat.

Some of the most common pharmacological treatments include antipsychotics, barbiturates, anticonvulsants, and muscle relaxers.

### SUMMARY

Implementations of the invention incorporate the concept of thermal biofeedback into the prosthetic socket. Embedded between a double layered socket is resistive heating wiring. When activated by the user, the socket begins to heat up. The heat stimulates the severed nerve endings in the residual limb, and forces the brain to focus on the heat, rather than to focus on pain signals being sent back to the brain.

When the user is wearing their prosthetic device and begin to feel an onset of phantom pain, they will activate the device using a controller, such as, but not limited to, a wireless remote. The user will then set their own heat setting, e.g., by selecting a desired or target temperature, based on the amount of the pain they are currently experiencing. In embodiments, this target temperature is transmitted wirelessly to the onboard micro controllers, which make all of the temperature adjustments and corrections inside of the socket.

The onboard micro controllers are housed in a unit over which the socket is mounted. The battery is housed in the same unit and weighs about five ounces, and has a fully charged battery life of up to six hours. It can be charged by placing the device on a solar charging mat.

The product can be easily assembled to fit all prosthetic types, as well as retrofitted to existing prosthetics. If a person's phantom pain is no longer an issue down the line, the product can be taken out of the prosthetic device.

It is an object of the invention to provide a pain reducing and eliminating prosthesis socket device that utilizes heat to reduce general and phantom pain.

It is also an object of the invention to provide a pain reducing and eliminating prosthesis socket device that incorporates an overall electrical circuit design and state of the art wireless control system to reduce general and phantom pain.

Implementations of the invention comprise and/or utilize a pain reducing and eliminating prosthesis socket device that provides heat to the point of contact between an amputee's body part and the prosthesis socket device that can eliminate or reduce general and phantom pain experienced by the amputee that would wear the prosthesis socket.

In a first aspect of the invention there is a pain free socket (PFS) system for a prosthetic device, the PFS system including: a temperature setting device configured to set a target temperature for the prosthetic device; an adjustable heating unit in a socket of the prosthetic device; a temperature sensor that detects an actual temperature in the socket of the prosthetic device; a controller that receives data from the temperature setting device and the temperature sensor, and that controls an amount of heat generated by the heating unit based on the data; and a display that displays the target temperature and the actual temperature.

In another aspect of the invention there is a method of providing heat to a socket of a prosthetic device. The method includes: detecting an actual temperature in the socket; obtaining a target temperature for the socket; selectively adjusting an amount of heat provided by a heating unit in the socket based on a difference between the actual temperature and target temperature; and displaying the actual temperature and target temperature on one of: a controller used to set the target temperature and the prosthetic device.

In another aspect of the invention, there is a method of manufacturing a prosthetic device having controlled heating, The method includes: installing an adjustable heating unit and a temperature sensor in a socket of a prosthetic device; providing a temperature setting device configured to set a target temperature; providing a controller that receives data from the temperature setting device and the temperature sensor, and that controls an amount of heat generated by the heating unit based on the data; and providing a display that displays the target temperature and the actual temperature.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 illustrates a front perspective view of a pain reducing and eliminating prosthesis socket device, in accordance with embodiments of the present invention.

FIG. 2 illustrates a cross-sectional perspective view along line 2-2 of FIG. 1, of a pain reducing and eliminating prosthesis socket device, in accordance with embodiments of the present invention.

FIG. 3 illustrates a diagram of a transmitter Fob and a receiver module of a pain reducing and eliminating prosthesis socket device, in accordance with embodiments of the present invention.

FIG. 4 illustrates a diagram of data transmitted byte types transmitted in the pain reducing and eliminating prosthesis socket device, in accordance with embodiments of the present invention.

FIG. 5 illustrates a diagram of a wireless processor printed circuit board components, in accordance with embodiments of the present invention.

FIGS. 6-8 show additional aspects of the pain free socket (PFS) and container in accordance with aspects of the invention.

FIGS. 9-11 show an external charging apparatus systems in accordance with aspects of the invention.

FIG. 12 shows an exemplary system in accordance with aspects of the invention.

FIGS. 13-15 show exemplary components that may be housed in the container and/or socket in accordance with aspects of the invention.

FIGS. 16-32 show exemplary modes of operation of the PFS in accordance with aspects of the invention.

FIG. 33 shows an embodiment in which the function and/or parts of the remote controller are incorporated into the container in accordance with aspects of the invention.

FIG. 34 shows an embodiment in which the function and/or parts of the remote controller are incorporated into a hand-held computing device in accordance with aspects of the invention.

FIG. 35 shows an embodiment in which the display is incorporated into the remote controller in accordance with aspects of the invention.

FIG. 36 shows an embodiment in which the components are incorporated into the prosthetic device in accordance with aspects of the invention.

FIG. 37 shows additional aspects of the invention.

### DETAILED DESCRIPTION OF EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

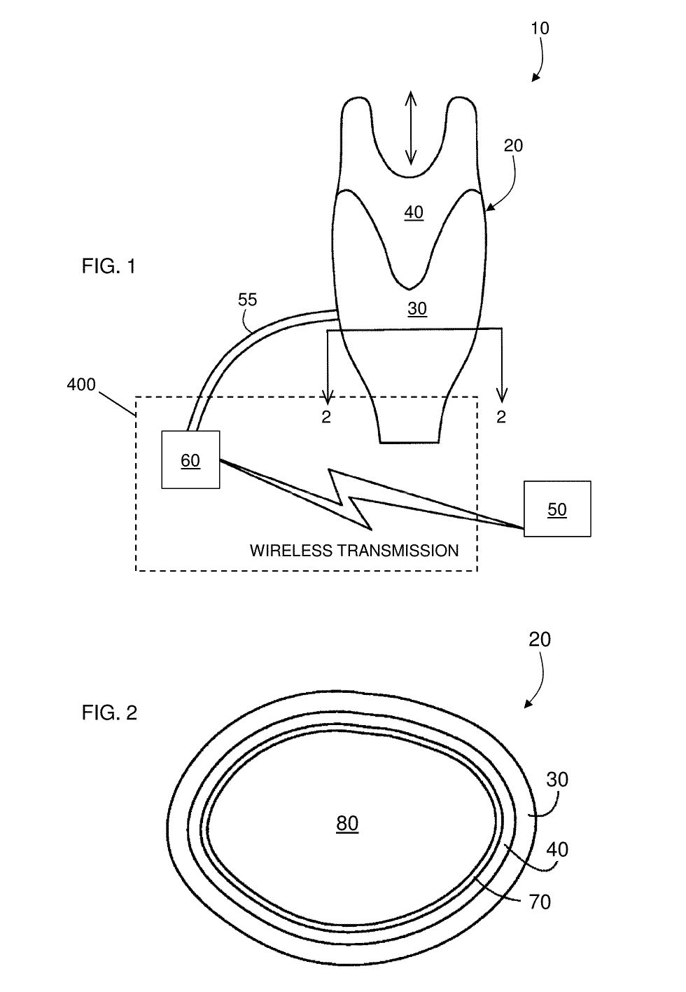

FIG. 1 is a front perspective view of a pain reducing and eliminating prosthesis socket device 10 for an amputee user with an amputated body part (not shown). The pain reducing and eliminating prosthesis socket device 10 is designed to be used as a below the knee device, although this is only one possible embodiment and the device 10 is not limited to just this embodiment. For example, aspects of the device 10 may be adapted for use with all prosthetic types, including but not limited to arms, AK/BK (above the knee/below the knee) legs, prosthetic feet/hands, etc. The pain reducing and eliminating prosthesis socket device 10 is made up of a prosthesis portion 20 that has a core socket 30 and a silicon socket cup 40 placed inside the core socket 30 to receive the user's amputated body part. The silicon socket cup 40 is set in-between the amputee's body part and the core socket 30. More details on the core socket 30 and socket cup 40 are discussed in the FIG. 2 description.

FIG. 1 also illustrates a wireless transmitter Fob 50 and a wireless receiver module 60 that houses some of the electronics components associated with the pain reducing and eliminating prosthesis socket device 10. These electronic components control some of the features of the pain reducing and eliminating prosthesis socket device 10. There is also an electronics cable or wiring 55 that transmits electrical information to and from the wireless receiver module 60 and serves as an electrical conduit between the wireless receiver module 60 and the prosthesis portion 20. There is also the wireless transmitter Fob 50 that is in wireless communication with the wireless receiver module 60. The wireless transmitter Fob 50 is physically small and wearable and is capable of transmitting serial data a short distance to the wireless receiver module 60 worn by the user amputee. The wireless transmitter Fob 50, the wireless receiver module 60 and the electrical and wireless components associated with the transmitter Fob 50 and the receiver module 60 are discussed in more detail herein. In accordance with aspects of the invention, the wireless receiver module 60 may be housed in a container 400 that is connected to the prosthetic device, as described in greater detail herein.

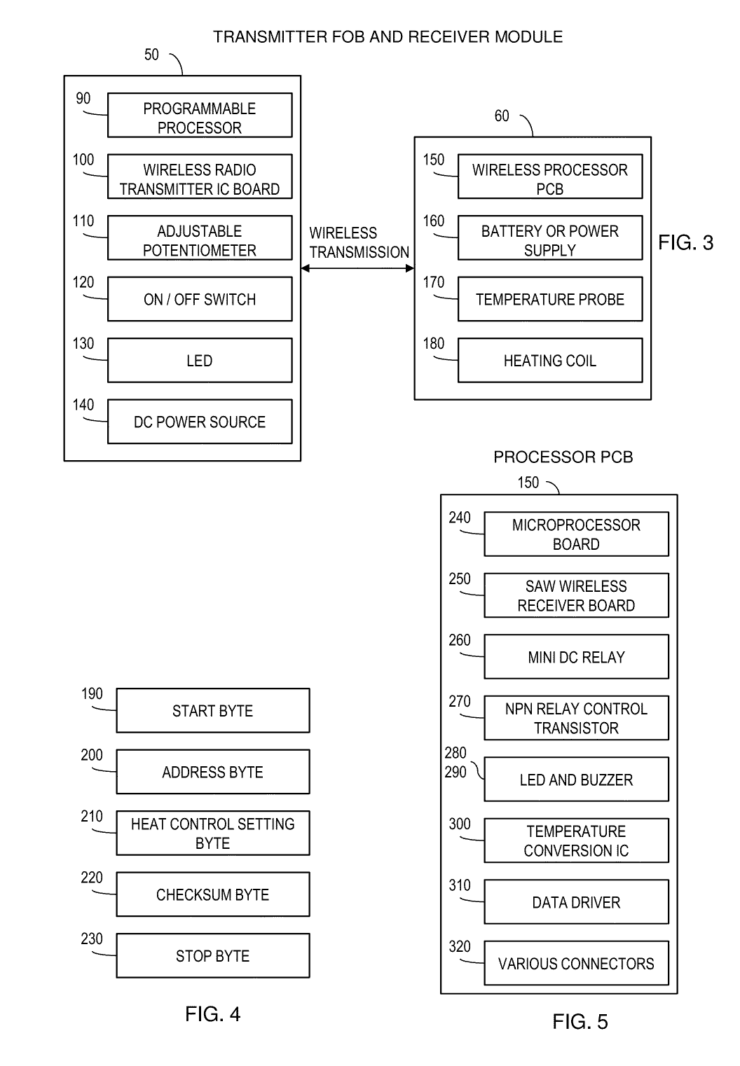

FIG. 2 illustrates a cross-sectional view of the prosthesis portion 20 of the pain reducing and eliminating prosthesis socket device 10. As previously indicated, the prosthesis portion 20 has a core socket 30, which is made of carbon fiberglass, and a socket cup 40, which is made of silicon. The core socket 30 may be made of carbon fiberglass because of the excellent insulating capabilities of carbon fiberglass, which is commonly used in other insulating applications. The socket cup 40 may be made of Pro-Flex, which is a silicone based material chosen for its high tolerance for heat, which is also used for other high heat tolerance applications. The invention is not limited to these materials and other materials may be used for the core socket and socket cup. The inner most layer of the prosthesis portion 20 is a laminate layer 70, which is between the socket cup 40 and the interior of the socket 80 or amputee's body part. A heating coil 180 (see FIG. 3 description) is also embedded between the socket cup 40 and the laminate 70 and allows the heating coil 180 to be installed easily. The laminate 70 is also thick enough to ensure an amputee user's comfort while being worn. The heating coil 180 is further discussed at much greater length in addition to the other electrical components of the pain reducing and eliminating prosthesis socket device 10 in the FIG. 3 description. The pain reducing and eliminating prosthesis socket device 10 is designed to address pain and specifically phantom pain with heat via the heating coil 180. However, the pain reducing and eliminating prosthesis socket device 10 can also include possible additional features such as a stump massage function, a stump skin care lotion dispenser or other external features (all of which are not shown) to further address an amputee user's comfort. Embodiments may comprise variable switches and resistors to incorporate a massage on/off switch, a massage intensity switch and massage duration timers (all of which are also not shown).

FIG. 3 illustrates the electrical and wireless components of the pain reducing and eliminating prosthesis socket device 10. As previously mentioned in the FIG. 1 and FIG. 2 descriptions, all of the wireless and electrical components associated with the pain reducing and eliminating prosthesis socket device 10 are utilized in wireless transmitter Fob 50 and the wireless receiver module 60. Both the wireless transmitter Fob 50, the wireless receiver module 60 and their electrical and wireless components are well known to persons who are skilled in the art.

In embodiments, the wireless transmitter Fob 50 includes a programmable microprocessor 90, a wireless radio transmitter IC board 100, an adjustable potentiometer 110, a momentary on and off switch 120, a light emitting diode 130 and a DC power source 140, although other components and/or configurations of components are contemplated within the scope of the invention. The wireless radio transmitter IC board 100 operates in the 434 MHz range, the adjustable potentiometer 110 is 5 kilohm and the power source is a 12 volt DC power source, although the wireless transmitter Fob 50 is not limited to the 434 MHz range, 5 kilohms and use of only a 12V DC power source, as other quantities can also be used. The components used to construct the transmitter fob 50 are currently available and are considered off-the-shelf electronic items.

The Fob's microprocessor 90 is the Arduino Mini Pro Board manufactured by Sparkfun Electronics, operating at 16 MHz and 5 volts DC, although the invention is not limited to this particular microprocessor any suitable microprocessor may be used within the scope of the invention. The indicator LED 130 and momentary switch (120) are very common. The microprocessor programming language is the open-source Arduino processing language. The transmitter IC board 100 is an industry-standard radio frequency (RF) SAW transmitter similar to those used in car alarms and keyless entry Fobs. This unit is made by the Holy Stone Enterprise Co., LTD. The invention is not limited to this particular transmitter IC board any suitable transmitter IC board may be used within the scope of the invention. For example, any suitable RF elements may be sued, and the invention is not limited to use of an RF module comprising a SAW filter.

The 5 kilohm slide or variable potentiometer 110 is available from multiple sources and can be a slide or round version, depending on availability and space requirements. The 12 volt DC power source 140 is a common, replaceable single N-size battery (battery not shown) and the LEDs 130 are switch and indicator LEDs and are very common components available from a variety of suppliers. The transmitter Fob 50 is also not limited to the brand names and makes indicated, as comparable electrical and wireless components can be used as well.

The momentary switch 120 connects the 12 volt power source 140 to both the transmitter Fob 50 and the microprocessor 90. It allows the microprocessor 90 to begin a boot process to start the onboard microprocessor functions. At this time, the microprocessor's onboard voltage regulator (not shown) reduces the 12 volts DC to a 5 volt DC supply and provides a regulated voltage from its 5 VDC output pin (not shown). The 5 volt supply is connected to one side of the adjustable potentiometer 110, which has 3 pins (not shown). The center pin of the potentiometer (not shown) is connected to the analog 0 pin of the microprocessor (not shown) to give a position or “sense” line. This input is an analog value that can range anywhere from 0 to 5 volts, depending on the resistive position of the potentiometer 110. The last or third pin of the potentiometer (not shown) is connected to a common ground (not shown) that returns the current to the negative side of the 12 volt power source 140. This common ground is also connected to the wireless radio transmitter IC board 100 and the microprocessor 90. After the microprocessor 90 boots into its operating mode, it checks the DC voltage on the Analog 0 pin and converts the analog voltage into a digital numerical value that can range from 0 up to 255, depending on the position of the slider arm of the potentiometer 110. This value is now the new heat control setting that will be transmitted to the wireless receiver module 60. The microprocessor 90 is now able to transmit a continuous stream of data to the receiver module 60. This pin is connected to the data pin (both pins not shown) of the wireless radio transmitter board 100. The data transmitted by the transmitter Fob 50 is made up of 5 data transmitted byte types of information, all of which are described in FIG. 4 and the FIG. 4 description.

The wireless receiver module 60 of the pain reducing and eliminating prosthesis socket device 10 includes an Arduino microprocessor board, a wireless processor printed circuit board (PCB) 150, a battery or power supply 160, a thermocouple temperature probe 170 and a heating coil 180. Details of the processor PCB 150 are depicted and discussed in FIG. 5 and the FIG. 5 description. The wireless receiver module 60 uses a standard lithium-polymer Li-Po battery pack (not shown) as the battery and power supply 160. The Li-Po battery pack is commonly found in radio-controlled toys and also serves as an 11.1 volt power supply for both the heating coil 180 and the wireless microprocessor board 150 of the pain reducing and eliminating prosthesis socket device 10. The Li-Po battery pack has the ability to supply a large amount of current to the heating coil 180 as needed. However, due to the weight of the Li-Po battery pack in its current configuration, it is more desirable and it is the best mode to use a custom, lightweight, moldable battery pack as the battery and power supply 160. Both types of battery packs are well-known to those schooled in the art and the pain reducing and eliminating prosthesis socket device 10 is not limited to using only these types of battery packs. The invention is not limited to this particular battery pack any suitable battery pack may be used within the scope of the invention.

A thermocouple temperature probe 170 is also utilized within the socket cup 40 of the pain reducing and eliminating prosthesis socket device 10. In embodiments, the thermocouple temperature probe 170 is a standard Type K chromel-alumel junction thermocouple used to measure temperature inside the socket cup 40, although any suitable temperature detecting device can be used with the invention. The pain reducing and eliminating prosthesis socket device 10 is not limited to using this type of thermocouple temperature probe 170. Multiple thermocouple temperature probes 170 can also be used to sense a larger area within the socket cup 40. The heating coil 180 used in the pain reducing and eliminating prosthesis socket device 10 is a piece of nickel-chromium wire (not shown) with a resistance that is low enough to heat quickly when power is applied to it. However, the nickel-chromium wire also has a small enough diameter to allow it to be woven into the silicone socket cup 40. The heating coil 180 also has a variable resistor (not shown) for specific heat point selection. This socket cup 40 with the heating coil 180, can be placed into the core socket 30 and prosthetic portion 20 without interfering with the amputee user's ability to use the pain reducing and eliminating prosthesis socket device 10. The heating coil 180 works based on the principles of thermal biofeedback from concentrated and controlled heat. The concentrated and controlled heat of the heating coil 180 works to stimulate severed nerve endings in the amputee body part and to force the brain to focus on the heat, rather than a missing body part that is no longer there. In embodiments, the heating coil 180 comprises an adjustable electrical resistive heating element that is operatively connected to and selectively controlled by the electronics associated with the wireless receiver module 60.

The wireless transmitter Fob 50 transmits wireless transmitter code information to the wireless receiver module 60 that is worn by the amputee, and the receiver module 60 decodes the code information to perform tasks from the wireless transmitter Fob 50. As depicted in FIG. 4, the wireless transmitter code information includes various data transmitted byte types such as a start byte 190, an address byte 200, a heat control setting byte 210, a checksum byte 220 and a stop byte 230.

The start byte 190 is a set of 8 bits of information that utilizes a string of alternating zeros and ones. This initial data string allows the wireless processor PCB 150 of the wireless receiver module 60 to begin locking onto the incoming wireless transmitted serial data stream, should a period of inactivity occur from the transmitter Fob 50, as will sometimes occur as a plan to save the battery life of the transmitter Fob 50. The address byte 200 is a preprogrammed address that will uniquely match the transmitter Fob 50 to its matched receiver module 60 if more than two pain reducing and eliminating prosthesis socket devices 10 operate in the same general area. This address byte 200 serves as a check on the receiving module 60 that will prevent one transmitter Fob 50 from accidentally sending wirelessly transmitted information to an unintended transmitter Fob 50 and pain reducing and eliminating prosthesis socket device 10. There is also a heat control setting byte 210 that indicates the digitized position of the heat control slider of the adjustable potentiometer 110. There is also a checksum byte 220, which is a numerical mathematical value generated by the transmitter programmable microprocessor 90 that allows the receiver module 60 to check if the address and heat control information received matches what was sent. The receiver module 60 gets the correct start byte 190 and the correct address byte 200 and will also verify a correct heat setting byte 210 that has been received by the use of the checksum byte 220. The stop byte 230 is a final byte in a transmitted serial data stream that lets the receiver module 60 knows it has reached the end of the serial data stream transmission and to stop processing any received serial data. The start byte 190, the address byte 200, the heat control setting byte 210, the checksum byte 220 and the stop byte 230 are all well known to those schooled in the art.

FIG. 5 illustrates the components of the wireless processor PCB 150 used in wireless receiver module 60 and the pain reducing and eliminating prosthesis socket device 10. In embodiments, the processor PCB 150 utilizes a microprocessor board 240, a SAW wireless receiver board 250, a mini DC relay 260, a NPN relay control transistor 270, a power indicator LED 280, a buzzer 290, a temperature conversion IC 300, a data driver IC 310 and various connectors 320 to connect the thermocouple temperature probe 170 and the heating coil 180 wiring leads. The wireless processor board 150 is the main board of the pain reducing and eliminating prosthesis socket device 10 that continuously monitors the core socket temperature and regulates the voltage across the heater coil 180 so that the heat produced by the heater coil 180 will closely match the heat control temperature settings received from the transmitter Fob 50. It also alerts the user if any new heat control temperature settings transmitted to the processor PCB 150 have been updated. The processor PCB 150 uses an Arduino Mini Pro microprocessor board that operates at 16 MHz and uses a 5 volt supply voltage in the pain reducing and eliminating prosthesis socket device 10. The SAW wireless receiver board 250 used with the processor PCB 150 operates in the 434 MHz range and an AN594 temperature conversion IC is the temperature conversion IC 300 used. The SAW wireless receiver board 250 used is made by the Holy Stone Enterprise Co., LTD. and the rest of the components used are available through multiple electronics parts vendors. The wireless processor PCB 150 however, is not limited to using the SAW wireless receiver board 250 made by the Holy Stone Enterprise Co., LTD. and is not limited to using an AN594 temperature conversion IC. Also the wireless processor PCB 150 is not limited to using an Arduino Mini Pro microprocessor board as its microprocessor board 240 as well.

In embodiments, in the wireless processor PCB 150 a 11.1 volt battery's positive and ground leads (not shown) are connected to the SAW receiver board 250 through a standard, polarized, removable 2-pin connector (not shown). This type of connector allows for the removal and charging of the battery separately from the SAW receiver board 250 as needed. The ground lead connects to a common ground bus (not shown) on the wireless processor PCB 150 and the positive lead connects to both the microprocessor's 240 raw vcc pin (not shown) and to one side of the 5 volt mini DC relay's 260 normal open pin contacts (not shown). The microprocessor 240 boots into its normal pre-programmed operation of sensing the core socket temperature and opening and closing the relay contacts (not shown) to keep the heating coil 180 at a nominal start up temperature of 90 degrees. The SAW wireless receiver board 250 gets its power from the regulated 5 volt supply pin and will only transmit data to the microprocessor 240 if it is received at the correct frequency. If no activity is sensed on the microprocessor 240 RX receiver pin (not shown), the microprocessor 240 will simply continue to regulate the temperature of the core socket 30 at the pre-programmed initial 90 degree setting. If new heat control settings are transmitted to the receiver module 60 at the correct frequency, the microprocessor 240 will begin to process the incoming serial data and look for the correct start byte 190, then the correct address byte 200, then the heat control setting byte 210, the checksum byte 220 and the stop byte 230. If the microprocessor 240 determines that either the start byte 190 or the address byte 200 is incorrect, it will discard the entire data string and wait for a new start byte 190. If the microprocessor 240 gets the correct start byte 190 and correct address byte 200, it will then verify that the correct heat control setting byte 210 has been received by use of the checksum byte 220. If everything is correct, then the microprocessor 240 will store the new heat control setting, send a short duration voltage pulse to the buzzer 290 indicating that the data was received correctly and then open or close the relay to adjust the socket temperature to the new heat setting. Once the wireless signal ends, the microprocessor 240 will continue monitoring the socket temperature and compare it to the new heat setting until new information is sent.

The wireless receiver board 250 cannot connect directly to the microprocessor board's RX input pin (not shown) due to the limits of its current sourcing capability. Therefore it is necessary to use a data driver IC 310 between the microprocessor board's RX pin and the data pins of the receiver board 250 (not shown). This data driver IC 310 is the 74LS02 Quad, two-input NOR gate. The wireless receiver board's data pins interface with the RX line of the microprocessor 240 by using two of the available NOR gates in series. It is necessary to use a temperature conversion IC 300 between the thermocouple temperature probe 170 and the microprocessor 240. The temperature conversion IC 300 used for this voltage conversion is the AD5895AQ. This is a temperature conversion IC 300 produced by Analog Device, Inc. The conversion IC 300 draws its 5 volt DC supply from the microprocessor's 5 volt pin. The conversion IC's ground pin (not shown) is tied into the common ground on the wireless control processor PCB (not shown). The output pin of the AD5895AQ is connected to the microprocessor's analog 0 pin (not shown). The microprocessor 240 uses onboard programming to use this voltage to compare the core socket temperature to the stored heat set point transmitted to it from the wireless transmitter Fob 50.

One of the microprocessor's digital I/O pins interfaces with the base of one NPN transistor 270 to control the current flow to the heating coil 180. The collector of the transistor (not shown) is connected to the 5 volt supply of the microprocessor 240 through a 1000 Ohm resistor (not shown). The emitter of the transistor (not shown) is then tied to the positive input side of the DC relay 260. The ground or negative side of the relay (not shown) is tied to the ground rail (not shown). The microprocessor 240 controls the status of the transistor 270 to make it act as an on/off switch. The relay 260 can then be pulsed on or off as determined by the microprocessor 240. The large current flow needed by the heating coil 180 can safely pass through the copper switches located inside the enclosed relay 260. The microprocessor on the receiver board 240 operates until the power supply is disconnected. If the power is disconnected, the pain reducing and eliminating prosthesis socket device 10 will revert to the low temperature setting at startup.

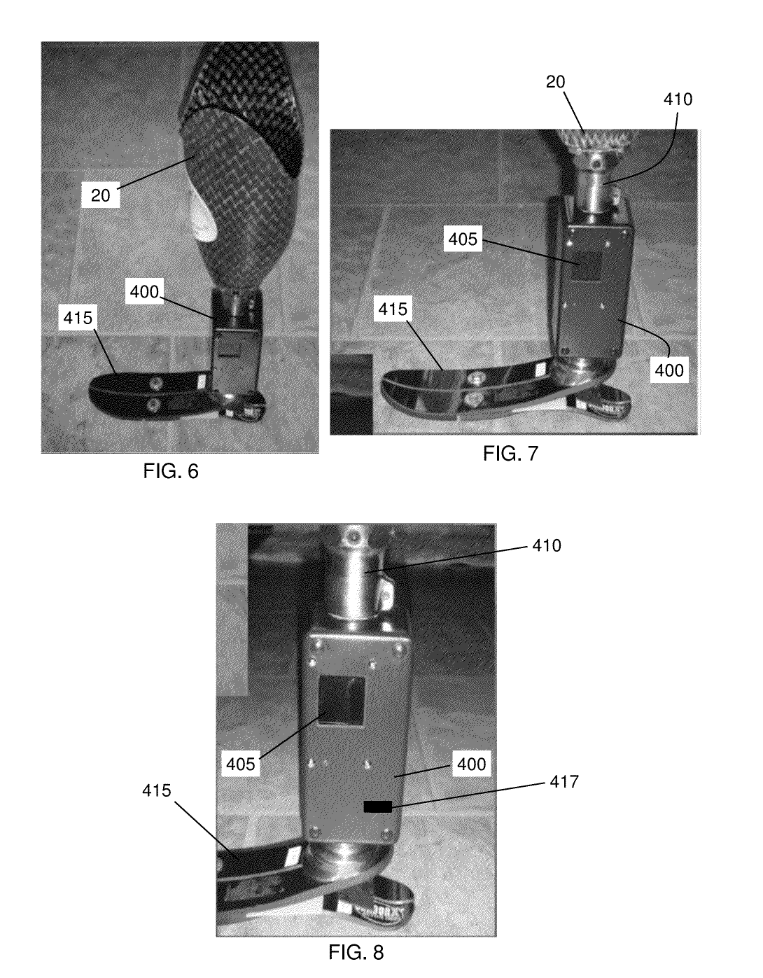

FIGS. 6-8 show additional aspects of the pain free socket in accordance with aspects of the invention. In embodiments, the components of the wireless receiver module, e.g., module 60, can be attached to the prosthetic device in a container 400. The container 400 may include a hole or transparent portion that accommodates a visual display 405 which is described in greater detail herein. The container 400 may be attached to the structural member 410 of the prosthetic device, e.g., between the socket 20 and an extremity portion 415 of the prosthetic device, e.g., a foot portion. The heating coil 180 and temperature probe 170 are contained in the socket 20 and are electrically connected to the wireless processor 150 and battery 160 that are housed in the container 400. In an exemplary embodiment, the container 400 comprises a box-like structure including separable portions that can be connected to one another around the structural member 410, i.e., with the structural member 410 passing through the container 400. In this embodiment, the structural member 410 comprises a post that extends between the socket 20 and the extremity portion 415.

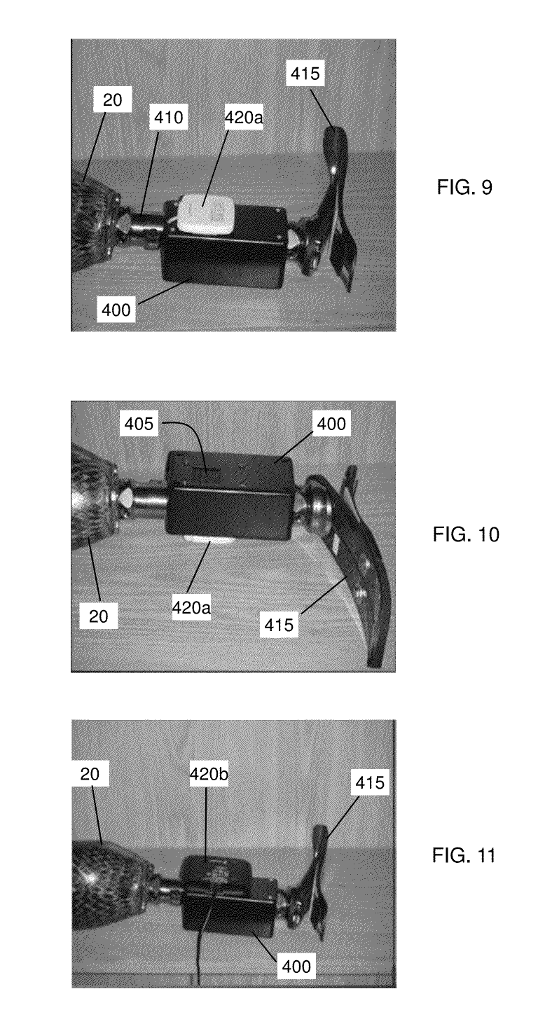

FIGS. 9-11 show an external charging apparatus 420a or 420b connected to the container 400 for charging the battery (e.g., battery 160) that is housed in the container 400 and that powers the heating coil 180 in accordance with aspects of the invention. The container 400 may have a charging port 417, such as a socket, plug, USB port, or any other suitable port that is configured to connect to an external electrical charging apparatus (e.g., as depicted in FIG. 8). The charging port 417 may be connected to the battery 160 for charging the battery 160 when an external electrical charging source is plugged into the charging port 417. Additionally or alternatively, the external charging apparatus may comprise a wireless charger that charges the battery 160 via electric and/or magnetic field.

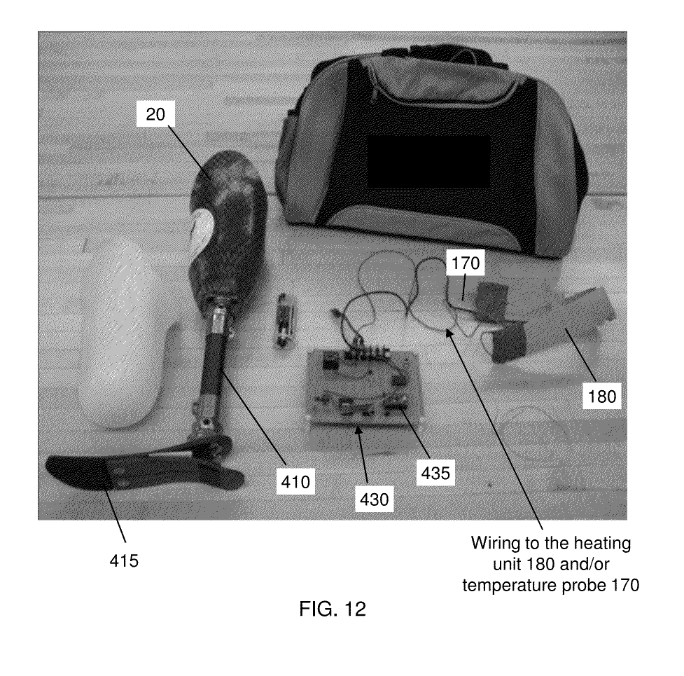

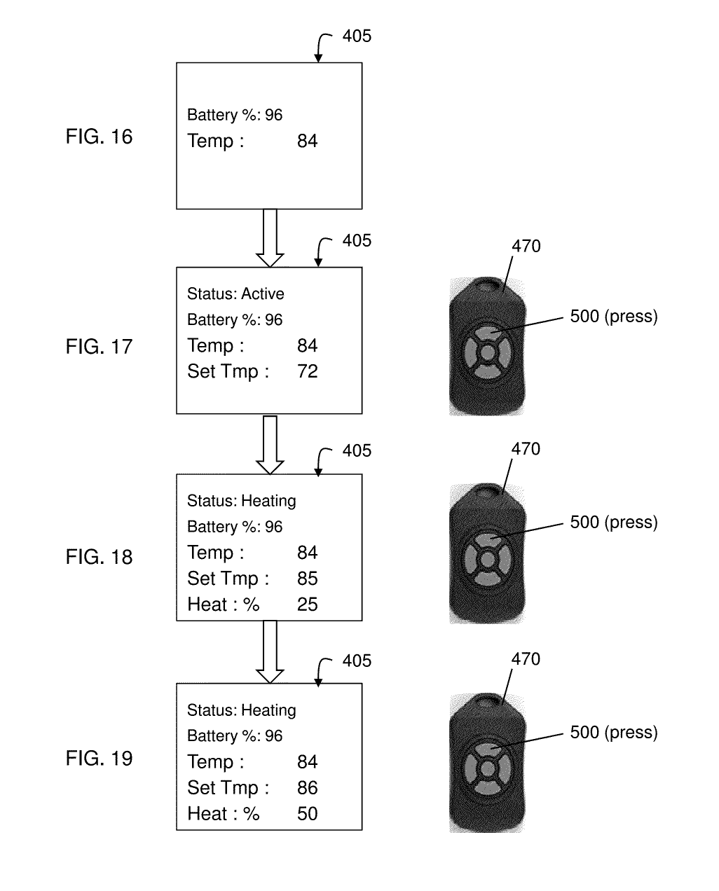

FIG. 12 shows an exemplary system in accordance with aspects of the invention including the socket 20, a structural member 410, and an extremity portion 415 of the prosthetic device without the container 400 attached. FIG. 12 also shows exemplary components that may be housed in the container 400 and/or socket 20 in accordance with an embodiment. For example, a controller 430 may be housed in the container 400 and include various electronics that are used in the pain free socket (PFS). The controller 430 may include some or all of the components described above with respect to the wireless receiver module 60, as well as other components. As shown in FIG. 12, the PFS system may include a temperature sensor (e.g., probe 170) for detecting an actual temperature in the socket, an adjustable heating unit (e.g., coil 180), and wiring (e.g., wiring 55) that operatively connects the temperature sensor and adjustable heating unit to the control electronics at the controller 430. In embodiments, the controller 430 receives data from the temperature sensor (e.g., actual temperature data from probe 170) and the temperature setting device (e.g., target temperature data from the remote controller 470), and controls the amount of heat generated by the adjustable heating unit (e.g., coil 180) based on the data (e.g., based on the target temperature and the actual temperature), e.g., in a manner similar to that described in FIGS. 1-5 and/or in FIGS. 16-31.

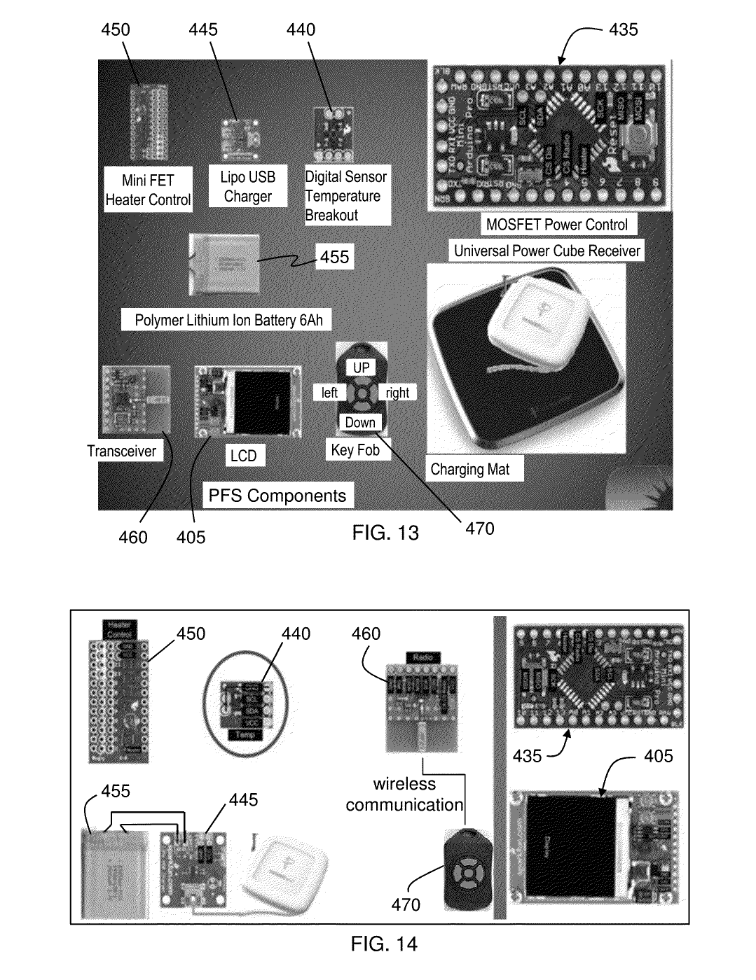

FIGS. 13-14 show exemplary components that may be included and used in controller 430 in a particular embodiment of the invention. For example, the controller 430 may include: a MOSFET power control 435 (e.g., a processor), digital sensor temperature breakout 440, Lipo USB charger 445, mini FET heater controller 450, polymer lithium ion battery 455 (which may correspond to or be the same as battery 160, or may be a different battery), and transceiver 460. The components shown in FIGS. 13-14 may be used with a PFS system such as that shown in FIG. 12 including a socket 20, a structural member 410, an extremity portion 415 of the prosthetic device, a container 400 (not shown), a temperature sensor (e.g., probe 170) for detecting an actual temperature in the socket, an adjustable heating unit (e.g., coil 180), and wiring (e.g., wiring 55) that operatively connects the temperature sensor and adjustable heating unit to the control electronics at the controller 430. In embodiments, the transceiver 460 communicates wirelessly with a remote controller 470 (e.g., a temperature setting device for setting a target temperature in the socket, which may correspond to the Fob 50 described earlier). In embodiments, the controller 430 receives data from the temperature sensor (e.g., actual temperature data from probe 170) and the temperature setting device (e.g., target temperature data from the remote controller 470), and controls the amount of heat generated by the adjustable heating unit (e.g., coil 180) based on the data (e.g., based on the target temperature and the actual temperature), e.g., in a manner similar to that described in FIGS. 1-5 and/or in FIGS. 16-31. As further shown in FIGS. 13-14, the PFS system may include the display 405, which may comprise an LCD, LED, or other type display device configured to display information according to the drawings and description herein.

The invention is not limited to a controller 430 including the specific components described with respect to FIGS. 13 and 14, and instead the controller 430 may include any suitable arrangement of components that provide the functionality described herein. As but one alternative example, the controller 430 may comprise a processor (e.g., CPU, etc.) and a memory device (e.g., RAM, etc.) that stores program code that, when executed by the processor, causes the PFS system to perform one or more of the functions described herein.

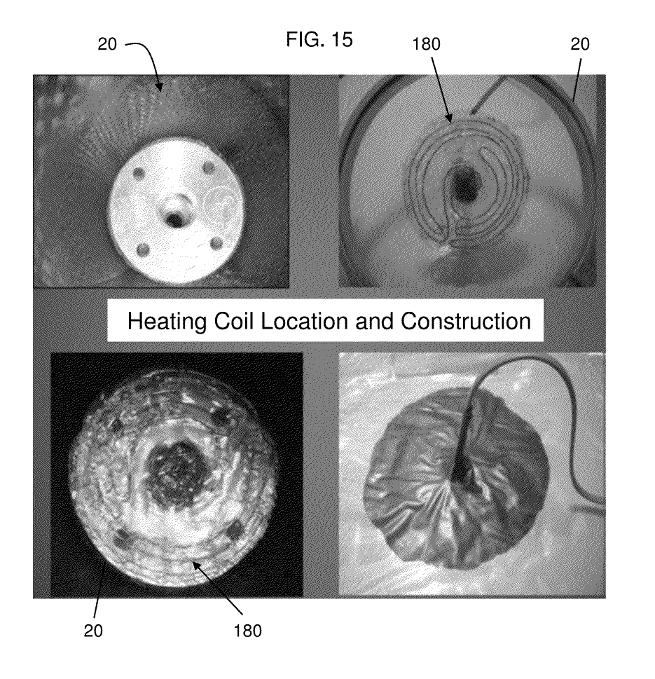

FIG. 15 shows exemplary location and construction of the heating coil 180 in the socket 20 in accordance with aspects of the invention. In some embodiments, the heating coil 180 is embedded in the socket 20.

FIGS. 16-31 show exemplary modes of operation of the PFS in accordance with aspects of the invention. In FIG. 16, the PFS is turned ON and the display 405 shows battery life (e.g., in % of battery remaining, % charge, etc.) and the actual temperature “Temp” sensed/detected in the socket, e.g., by temperature probe 170. When the user presses a first button 500 on the remote controller 470 (e.g., the top button), the controls set the status to active and the display 405 shows a target or desired temperature, e.g., as “Set Tmp”, as shown in FIG. 17. The user may incrementally change (increase) the value of Set Tmp by pressing the first button. Each button press corresponds to a predefined incremental change in the Set Tmp, e.g., one degree F. (Fahrenheit).

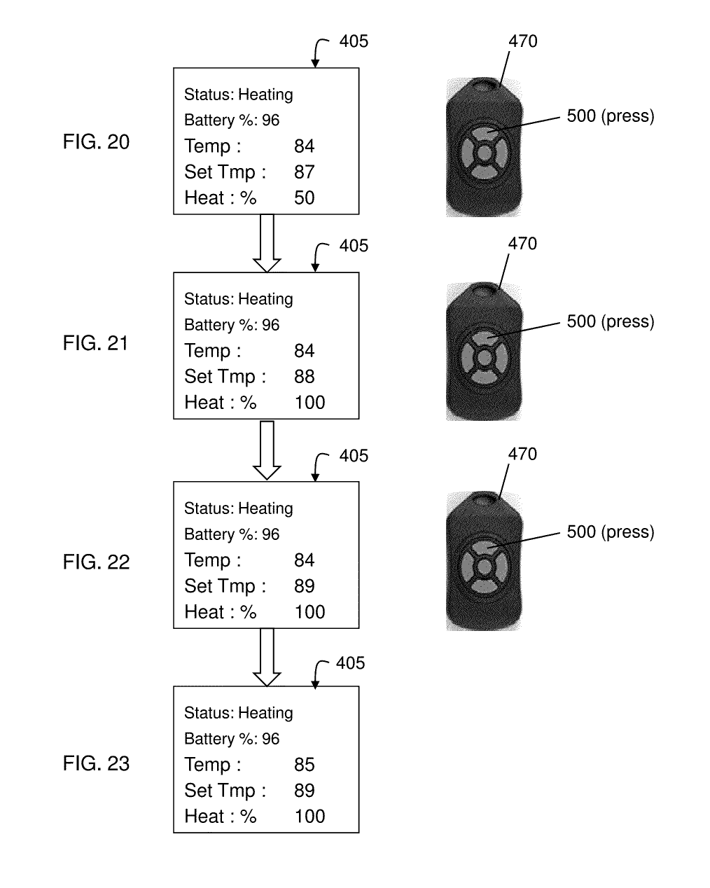

As shown in FIG. 18, when the target value (Set Tmp) exceeds the actual value (Temp), then the status changes to heating and the display 405 indicates an amount of heating (Heat %). The target temp continues to increase as the user continues to press the first button, as shown in FIGS. 19-22. The “Heat %” represents the effort being exerted by the heater (e.g., coil 180) to reach the target temperature. The amount of heat may be predefined with the logic of the electronics to any desired configuration. For example, a delta T of 1 degree between Temp and Set Tmp (e.g., FIG. 18) may result in 25% heat (e.g., causing the heating coil 180 to operate at a 25% heating level); a delta T of 2 or 3 degrees between Temp and Set Tmp (FIGS. 19 and 20) may result in 50% heat (e.g., causing the heating coil 180 to operate at a 50% heating level); and a delta T of 4 or more between Temp and Set Tmp (FIGS. 21 and 22) may result in 100% heat (e.g., causing the heating coil 180 to operate at a 100% heating level). These values are merely exemplary, and any desired predefined heating configuration may be used based on the difference (delta T) between the actual and target temperatures (e.g., between Temp and Set Tmp)

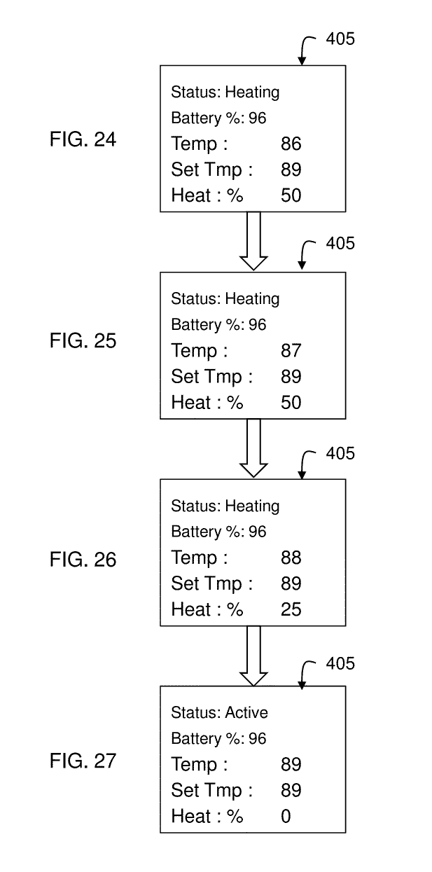

FIG. 23 shows that the actual temperature is detected as rising from 84 to 85 as a result of the heating coil 180 producing heat in the socket 20. FIGS. 24-27 show how the actual temperature continues to rise based on the heating, and also show how the heat % changes based on the difference between the actual (Temp) and target (Set Tmp). As shown in FIG. 27, when the actual reaches the target, then heat % goes to zero. In this manner, the PFS attempts to maintain the actual temperature equal to the target temperature by turning the heater coil 180 on and off based on the difference between the actual (Temp) and target (Set Tmp).

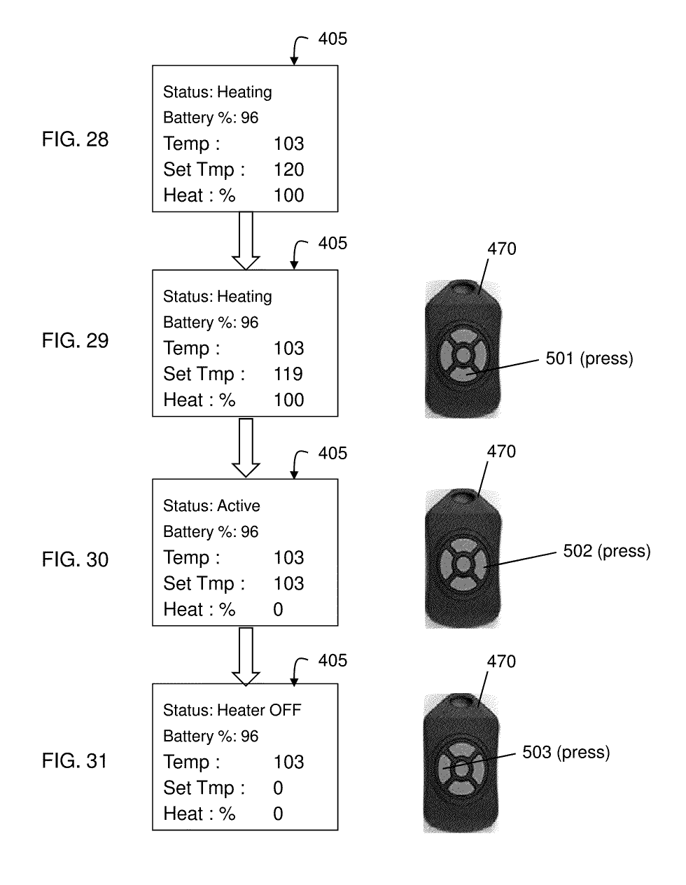

As shown in FIG. 28, the target temperature (Set Tmp) may have a maximum value, such as 120 degrees F. The invention is not limited to this value and any suitable maximum may be programmed into the electronics.

FIG. 29 depicts the system user pressing a second button (e.g., the bottom button) 501 on the remote controller 470. When the user presses the second button, the target temperature (Set Tmp) is incrementally decreased, e.g., by one degree F. per push of the button 501. If the user decreases the target to less than the actual, then the heater coil 180 will be turned off.

FIG. 30 depicts the system when the user presses a third button (e.g., the right button) 502 on the remote controller 470. In this case, the target (Set Tmp) is set equal to the actual (Temp). The heat % goes to zero, since the target and actual are equal, and the system attempts to maintain the target temperature by appropriately turning the heater coil 180 on and off based on the difference between the actual and the target (e.g., the detected actual temperature may decrease over time, at which point the system will turn the heater coil 180 back on). The system will stay in this mode after pressing the third button 502 until another button is pressed.

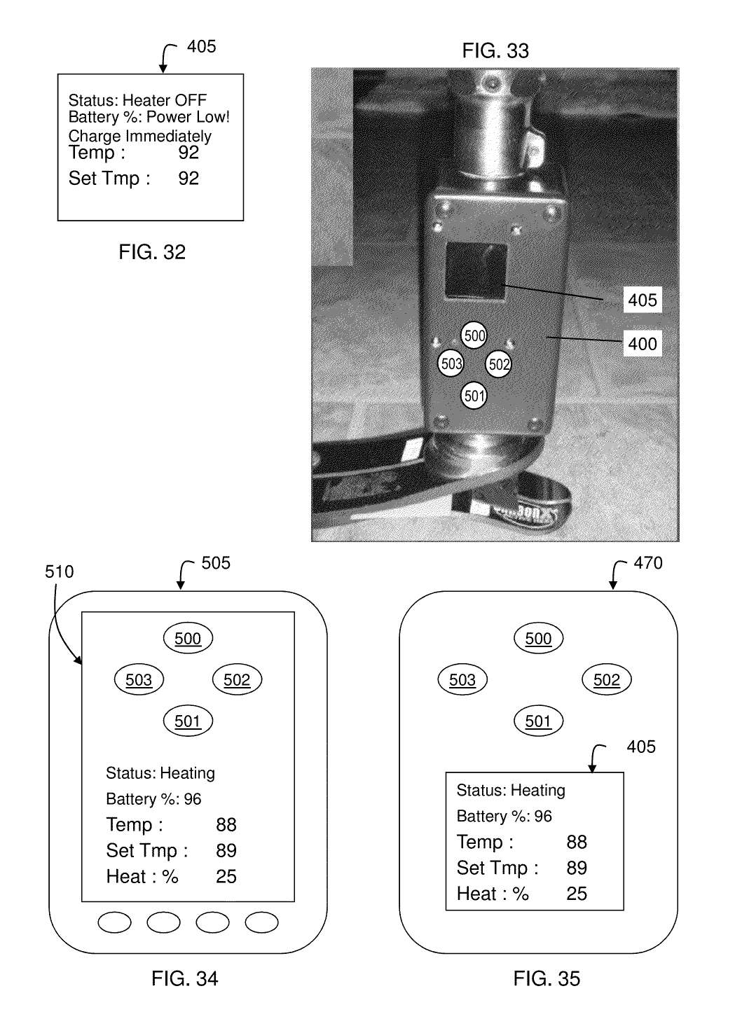

FIG. 31 depicts the system when the user presses a fourth button (e.g., the left button) 503 on the remote controller 470. In this case, the target (Set Tmp) is set to zero, and the heater coil 180 is turned off. As shown in FIG. 32 the system may provide a low battery warning via the display 405.

FIG. 33 shows an alternative embodiment in accordance with aspects of the invention. The function and/or parts of the remote controller 470 (e.g., Fob 50) may be incorporated into the container 400. For example, rather than having a separate remote controller 470 (e.g., Fob 50), the buttons 500, 501, 502, and 503 may be incorporated into the container 400 and operatively connected (e.g., wired) to the controller housed in the container 400. In this embodiment, the system operates in the same manner as described with respect to FIGS. 16-32 when the user oppresses the various buttons 500, 501, 502, and 503. In this manner, the separate remote controller 470 (e.g., Fob 50) can be eliminated by providing the user controls (e.g., the buttons 500-503) on the container instead of on a separate remote controller 470.

FIG. 34 shows an alternative embodiment in accordance with aspects of the invention. In this embodiment, the functions of the display (e.g., display 405) and the remote controller (e.g., remote controller 470) are embodied in a hand held computing device 505, such as, but not limited to, a smart phone, a personal digital assistant (PDA), tablet computer, notebook computer, etc., that comprises a visual display (e.g., LCD screen), processor or microprocessor, wireless transmitter, and I/O device. For example, a software application that is stored on the computing device 505 and executed by a processor of the computing device 505 may cause a display 510 of the computing device 505 to show: (i) the information previously described with respect to display 405 (e.g., Status, Battery %, Temp, Set Tmp, Heat %, etc.), and (ii) the visual representations of buttons 500, 501, 502, and 503. The user may manipulate the buttons 500, 501, 502, and 503 using the I/O device of the computing device 505 (e.g., touch screen, cursor, etc.), and the computing device 505 may wirelessly transmit data signals to the receiver (e.g., wireless receiver 60, transceiver 460, etc.) to selectively control an amount of heating provided by the heating coil 180 in the socket 20. In an exemplary embodiment, the computing device 505 comprises a smart phone having a touch screen display 510, and the user controls the heating coil 180 by pressing the various visual representations of the buttons 500-503 on the touch screen display 510. In this embodiment, a software application running on the smart phone is configured to accept the user input via the touch screen display 510 and accordingly send control signals to the (e.g., wireless receiver 60, transceiver 460, etc.) to control the heating coil 180 based on such input in a manner similar to that described with respect to FIGS. 16-32. In this embodiment, the software application running on the smart phone is also configured to update the display information 405 (e.g., Status, Battery %, Temp, Set Tmp, Heat %, etc.) in a manner similar to that described with respect to FIGS. 16-32.

FIG. 35 shows an alternative embodiment in accordance with aspects of the invention. In this embodiment, the display 405 is incorporated into the remote controller 470 (e.g., Fob 50) in addition to or alternatively to being in the container 400. Suitable hardware and software may be incorporated into the remote controller 470 to control the display 405 in a manner similar to that described with respect to FIGS. 16-32. In this manner, the user may view the display 405 on the remote controller 470 (e.g., Fob 50) when they are pressing the buttons 500, 501, 502, and 503 on the remote controller 470 (e.g., Fob 50).

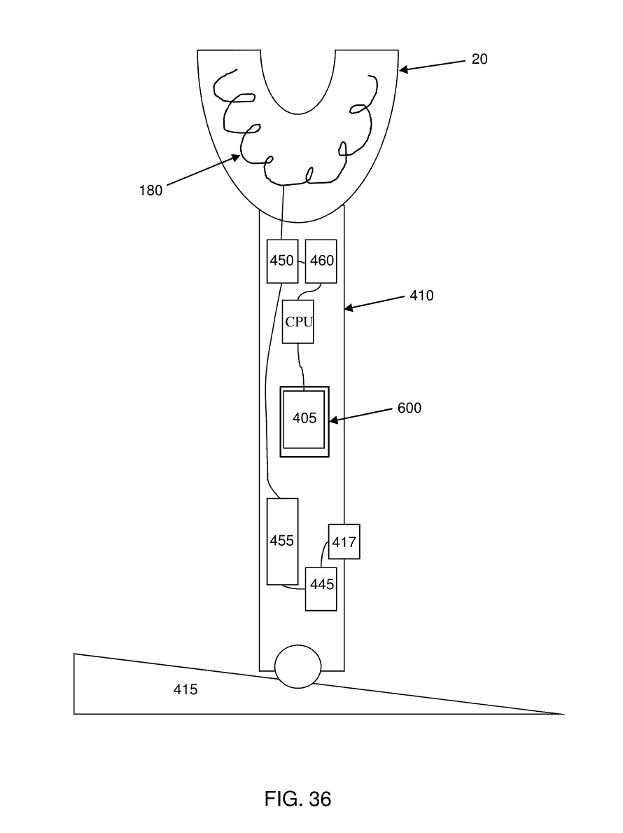

FIG. 36 shows an alternative embodiment in accordance with aspects of the invention. In this embodiment, the display 405 and some or all of the components of the PFS (e.g., components 445, 450, 455, 460, etc.) are contained within the socket 20 and/or structural member 410 of the prosthetic device. In this manner, the components of the PFS can be hidden from view, and the container 400 that is attached to the exterior of the structural member 410 can be omitted. A charging port 417 can be provided through the structural member 410 for providing access to charge the battery 455 (e.g., battery 160). The display 405 can be made visible through a transparent portion of the member 410, or a hole may be provided in the member 410 for viewing the display 405, the transparent portion/hole being indicated at number 600.

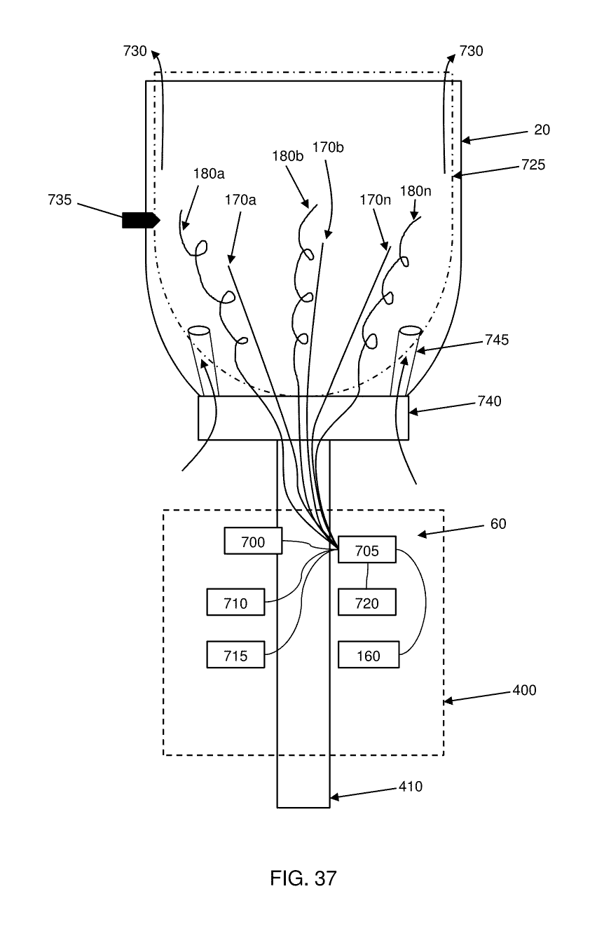

FIG. 37 shows additional aspects of the invention that may be used in conjunction with any of the embodiments described herein. In one additional aspect, a vibration element 700 is arranged within or on the socket 20, container 400, or structural member 410 and operatively connected to a controller 705 (e.g., a processor in the wireless receiver module 60 or controller 430). In embodiments, the controller is configured to monitor a charge level of the battery 160 (e.g., percent of battery life remaining) and send a control signal to cause the vibration element 700 to vibrate when the charge level of the battery 160 falls below a predefined threshold value (e.g., 20% battery charge remaining). The vibration element 700 may comprise any suitable mechanism for causing a vibration, such as an eccentrically weighted motor or other suitable mechanism. In this manner, a user wearing the socket 20 may be informed via vibration of the vibration element 700 when the battery 160 is running low on its charge.

In another additional aspect depicted by FIG. 37, the battery 160 includes a motion-rechargeable battery that harnesses kinetic energy of movement of the socket 20 to recharge itself. The technology behind motion-rechargeable battery is known such that further explanation is not necessary here. In embodiments, the battery 160 includes a motion-rechargeable battery, and the motion of the socket 20 that results from the user moving (e.g., walking) is used to recharge the battery 160.

In a further additional aspect depicted by FIG. 37, a controller 705 (e.g., a processor in the wireless receiver module 60 or container 400) is configured to track and measure steps taken by a user wearing the prosthetic device containing the socket 20, track and measure miles walked, and track and measure stride length. In embodiments, the PFS is provided with a GPS (global positioning system) unit 710 and/or an accelerometer 715, and the controller executes a software application that utilizes data from the GPS and/or accelerometer to function as a pedometer. In this manner, the controller in the PFS may obtain pedometer data such as: track and measure steps taken by a user wearing the socket 20, track and measure distance walked by a user wearing the socket 20, and track and measure stride length of a user wearing the socket 20. The controller may store the pedometer data in a memory unit 720 (e.g., RAM, etc.) contained in the PFS (e.g., in the container 400), and the pedometer data may be transmitted from the memory unit to an external computer device (e.g., a smart phone, tablet computer, laptop computer, desktop computer, etc.) wirelessly or using a wired connection (e.g., USB, etc).

In an even further additional aspect depicted by FIG. 37, the controller 705 is configured to track and measure thermal biofeedback results of the PFS. In embodiments, the controller stores a history of the actual temperature (e.g., “Temp” detected by probe 170) and/or target temperature (e.g., “Set Tmp”) and/or heating power (e.g., “Heat %”) versus time (e.g., year, month, day, hour, second, etc.). The controller may store the historical thermal biofeedback data in a memory unit 720 (e.g., RAM, etc.) contained in the PFS (e.g., in the container 400), and the historical thermal biofeedback data may be transmitted from the memory unit to an external computer device (e.g., a smart phone, tablet computer, laptop computer, desktop computer, etc.) wirelessly or using a wired connection (e.g., USB, etc). The historical thermal biofeedback data may be displayed, e.g., as a graph of temperatures versus data and time, e.g., for treatment, education, etc.

In an additional aspect depicted by FIG. 37, the PFS may contain a sweat-wicking material 725 in the socket 20 that suctions perspiration out of the socket 20. The sweat wicking material 725 may utilize directional capillary action that causes perspiration to move through the material in a direction toward the outside of the socket 20, as indicated by arrows 730. The sweat-wicking material 725 may be placed directly against the skin of the user in the socket 20, between the skin and the heating coil 180.

In another additional aspect depicted by FIG. 37, the PFS may contain an injection system 735 connected to the socket 20 to deliver injections from the prosthetic device. The injection system may include any suitable injector, such as a needle injector or a needle-free jet injector. The injection system may include a reservoir having a single dose or plural doses, or may utilize cartridges for dosing the medication that is applied via the injection. The injection system may be manually operated or automated. The injection system may be used to deliver any desired medication (e.g., pain reliever, antibiotic, anti-inflammatory, etc.) to the body part of the user that is contained in the socket 20 while the user is wearing the prosthetic device.

In another additional aspect depicted by FIG. 37, the PFS may contain an internal fan/ventilating system 740 connected to the socket 20 to deliver cooling air to the interior of the socket 20. In embodiments, the fan 740 is an electric fan that is powered by the battery 160 (or a separate power supply) and is turned on and off by the controller 705 (or a separate manual on/off switch mounted on the fan 740 and/or socket 20). In embodiments, the fan 740 moves air from outside the socket 20 to the interior of the socket 20 via at least one flow path 745 (e.g., conduit) in the socket 20. In a particular exemplary embodiment, the fan 740 is at the base of the socket 20 and forces air through a plurality of passages to a plurality of locations internal to the socket 20.

In another additional aspect depicted by FIG. 37, the heating coil 180 contains plural portions 180a, 180b, . . . , 180n that are each connected to the heater control (e.g., heater controller 450) and that are individually controllable to selectively apply heat in varying amounts to different locations within the socket 20. Each portion 180a, 180b, . . . , 180n may be provided with a respective temperature probe 170a, 170b, . . . , 170n in the same spatial region of the socket 20. In this manner, a user may set different target temperatures (e.g., Set Tmp) for each different region of the socket, and the PFS will individually control each heating coil portion 180a-n in a manner similar to that described with respect to FIGS. 16-31. In embodiments, the remote controller 470 has a selector (e.g., a button, dial, switch, etc.) to designate which heating coil portion 180a-n is being adjusted when the user presses buttons 500-503. In other embodiments that utilize the computing device 505 as the remote control, the computing device 505 may be programmed to display visual indications of the different regions of the socket 20 associated with the different heating coil portions 180a-n, and permit the user to select which heating coil portions 180a-n to control via touch screen input.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A pain free socket (PFS) system for a prosthetic device, comprising:
a temperature setting device configured to set a target temperature for the prosthetic device;
an adjustable heating unit in a socket of the prosthetic device;
a temperature sensor that detects an actual temperature in the socket of the prosthetic device;
a controller that receives data from the temperature setting device and the temperature sensor, and that controls an amount of heat generated by the heating unit based on the data; and
a display that displays the target temperature and the actual temperature.

2. The system of claim 1, wherein the controller is housed in a container that is attached to the prosthetic device.

3. The system of claim 2, wherein the display is in the container.

4. The system of claim 2, wherein the temperature setting device is in the container.

5. The system of claim 2, wherein the temperature setting device is in a remote controller that is separate from the container and which communicates wirelessly with the controller.

6. The system of claim 5, wherein the display is in the remote controller.

7. The system of claim 1, wherein the temperature setting device comprises inputs that at least one of: increment the target temperature, decrement the target temperature, set the target temperature equal to the actual temperature, and turn off the heating unit.

8. The system of claim 1, wherein the temperature setting device and the display are incorporated into a hand held computer device that communicates wirelessly with the controller.

9. The system of claim 8, wherein the hand held computer device runs an application that provides a user with a visual display of controls that permit the user to at least one of: increment the target temperature, decrement the target temperature, set the target temperature equal to the actual temperature, and turn off the heating unit.

10. The system of claim 1, wherein the temperature setting device, the controller, and the display are mounted inside at least one of the socket and a structural member of the prosthetic device.

11. The system of claim 1, further comprising a vibration element connected to the prosthetic device that vibrates when a charge of a battery associated with the heating unit falls below a predetermined threshold value.

12. The system of claim 1, wherein:
the heating unit comprises a plurality of separate heating units arranged in different locations in the socket;
the temperature sensor comprises a plurality of separate temperature sensors corresponding to respective ones of the plurality of separate heating units; and
the controller controls each one of the plurality of separate heating units separately.

13. The system of claim 12, wherein the temperature setting device is configured to set a plurality of set a target temperatures corresponding to respective ones of the plurality of separate heating units.

14. The system of claim 1, further comprising a global positioning system (GPS) unit and/or an accelerometer connected to the prosthetic device, wherein the controller uses data from the GPS unit and/or accelerometer to generate pedometer data.

15. The system of claim 1, wherein the controller stores historical thermal biofeedback data of the target temperature and the actual temperature.

16. The system of claim 1, further comprising a fan connected to the socket, wherein the fan forces air from outside the socket to inside the socket.

17. The system of claim 1, further comprising a sweat-wicking material inside the socket that wicks moisture from inside the socket to outside the socket.

18. The system of claim 1, further comprising an injection device connected to the socket that is configured to provide an injection to a body part of a user contained inside the socket.

19. A method of providing heat to a socket of a prosthetic device, comprising:
detecting an actual temperature in the socket;
obtaining a target temperature for the socket;
selectively adjusting an amount of heat provided by a heating unit in the socket based on a difference between the actual temperature and target temperature; and
displaying the actual temperature and target temperature on one of: a controller used to set the target temperature and the prosthetic device.

20. A method of manufacturing a prosthetic device having controlled heating, comprising:
installing an adjustable heating unit and a temperature sensor in a socket of a prosthetic device;
providing a temperature setting device configured to set a target temperature;
providing a controller that receives data from the temperature setting device and the temperature sensor, and that controls an amount of heat generated by the heating unit based on the data; and
providing a display that displays the target temperature and the actual temperature.
